(19) United States
(12) Patent Application Publication
(10) Pub. No.: US 2003/0190675 A1
(43) Pub. Date: Oct. 9, 2003

(54) Method and functional particles for carrying out chemical or biological reactions or syntheses

(76) Inventor: Andreas Schober, Schwangau (DE)

Correspondence Address: JORDAN AND HAMBURG LLP, 122 EAST 42ND STREET, SUITE 4000, NEW YORK, NY, 10168, US

(21) Appl. No.: 10/363,067

(22) PCT Filed: Aug. 30, 2001
(86) PCT No.: PCT/EP01/10033

(30) Foreign Application Priority Data
Aug. 30, 2000 (DE) 10043369.3

(51) Int. Cl. G01N 33/53; G01N 33/543

(52) U.S. Cl. 435/7.1; 436/518

(57) ABSTRACT

The invention relates to a method and functional particles for carrying out chemical or biological reactions or syntheses. The aim of the invention is to provide a solution ensuring complete compatibility between micro and macroscales, whereby a large variety of coupling reactions can be carried out, and a clear allocation of the reaction or synthesis products to the individual functional particles is ensured, entailing little effort. In order to achieve this, x charges of functional particles are provided for n reaction solutions, x being equal to or less than n, the density of said particles being variably determined in such a way that they can be separated according to the density thereof even when being charged with the complete reaction or synthesis product.

### BACKGROUND OF THE INVENTION

The invention relates to a method and to functional particles for carrying out chemical or biological reactions or syntheses which particularly are used in the automated laboratory work in the field of combinatorial chemistry and molecular biotechnology.

Functional spheres, beads, are already introduced as supports for chemical syntheses. Such particles (beads) are made, for example, of glass or polystyrene. Solid phase syntheses on beads are already introduced in the oligonucleotide chemistry and in the peptide chemistry as well as in the synthesis of organic molecules on a macroscopic scale. Hereby the solid phase support will be filled into columns or plates and will be flushed with a reagent, wetted and washed and the chemical synthesis will be induced in accordance with the synthesis protocol and the employed synthesis device, respectively.

Also in the biochemical research solid phase supports (for example the so-called Dynabeads of the Dynal Company) are already introduced as supports for biochemical material. Thereby, the supporting material is coated, for example, with strept-avidin which permits to bind biotinized RNA or DNA-sequences to such supports via a strept-avidin biotin binding. Thus it is possible to carry out binding experiments in a selective manner by washing the beads with the target searched for.

The chemical synthesis itself will be realized out on the sphere, the “bead”, in such a manner that the initial compound is covalently bonded to a so-called “linker” (a coupling molecule to the solid phase), whereby the initial compound can be converted by reagents in accordance with the synthesis protocol and, if required, separated from the linker. Depending on the realisation, the coupling molecules can be phospho-amidite in the oligonucleotide chemistry or the amino acids in the peptide chemistry (Merryfield synthesis). The advantage of this method lies in its capability to provide a large excess of reagents as well as to iterate the coupling, washing, and de-protection processes as often as desired.

It were these advantages that such methods were also used in the organic chemical synthesis where, in a same manner, the molecule to be coupled was synthesized to the solid phase via a linker.

The search for novel compounds having desired functions is increasingly supported by non-rational methods of the synthesis of leading structures in order to synthesize a great number of diverse structures, to begin with. This applies for the synthesis of small molecules by chemical or photosynthetic procedures as well as for the synthesis of larger molecules such as peptides, proteins, and nucleinic acids under utilization of chemical or enzymatic reaction processes. By the availability of miniaturized analysis procedures which permit to detect the so-called fitness (the quality of the chemical or biological properties of the product of a synthesis related to at least one desired function and property) and to quantitatively measure the same, respectively, the synthesis of such leading structures on a miniaturized scale becomes possible, whereby, however, a quantitative product analysis can, in most cases, only be carried out afterwards by a post-analysis of the product in greater quantities.

A limiting factor as to the above described introduced synthesis procedures is that the availability and the synthesis, respectively, of a great number of diverse molecules are limited in number.

The chemical synthesis is carried out in the commercial synthesizers on such functional spheres either in columns or in a planar manner via pipetting robots in microtiter plates. Hence, the degree of parallelizing does not substantially exceed the degree of the up to now common microtiter standards of 96 diverse samples. The present day automation in laboratories in the field of synthesis enables a degree of parallelizing of some hundred samples (for example, Chem Speed 384) in a volume range of from about 100 μl up to some ml (refer to, for example, DeWitt, A. W. Czarnik: Automated synthesis and Combinatorial chemistry Current opinion in Biotechnology 1995, 6: 640-645).

The synthesized substances are present in macroscopic amounts per column. However, the parallelity in dependence on the machinery does not suffice to attain a combinatorial variety, not even in approximation, (for example, oligonucleotide alphabet: 4ⁿ, peptide alphabet: 20ⁿ, organic libraries (α)ⁿ. The oligonucleotide strands of the length 6 sum up to a variety of about 4000 strands, oligopeptides of the length 6 already yield 64 million of different substances).

The approach known from the UHTS (ultrahigh throughput screening) relates to the carrying out of automated miniaturised screening systems (an overview is given in: Pauwels, H. Azijn, MP. de B, C. Claeys, K. Herzog “Automated techniques in biotechnology” Current opinion in Biotechnology 1995, 6: 111-117, or Zhao, H., Arnold F. H. “Combinatorial protein design: Strategy for Screening Protein Libraries”, 1997 Current opinion in Structural Biology, Vol. 7 pp. 480-485, Burbaum, J. J., Sigal, N. M. “New technologies for High-Throughput Screening” (1997) Current opinion in Chemical Biology, 1:72-78. Commercially this kind of approach was introduced in particular by the firms Evotec, Hamburg and Aurora, San Diego. Hence, the need for great substance libraries, which can be fed into screening lines for testing, has considerably increased.

Another way for generating great molecule libraries is taken by the so-called “Split and Pool” or “Split and Mix” methods (see Lam et al. (1991) Nature 354:82, Glaser et al. (1992) J. Immunol, 149:3903-3913, Lam et al. (1993) Bioorg. & Med. Chem. Lett. 3:419 and Sebestyen et al. (1993) Bioorg. & Med. Chem. Lett. 3:413).

This method couples respective different monomers in respective n synthesis columns. Then these mixtures are “pooled”, that is, mixed, in new reaction chambers, and then distributed in a defined manner to the respective columns and the different coupling steps will be executed.

The advantage of this method lies in its capability to synthesize a broad range of different varieties whereby, however, it has to be ensured on the side of the procedure technology that exactly one substance has to be generated on each bead, which can be chemically uniquely characterized. A disadvantage consists in that it is initially not clear which substance is to be found on which bead. This problem, however, is not relevant in the first step of a binding experiment. Only selected variants have to be characterized.

In order to maintain the general applicability of the method, work is under progress to carry out the characterization in an indirect manner by coding or in a direct manner by analysis procedures such as MS and MAS-NMR or IR-spectroscopy (W. L. Fitch, G. Detre, C. P. Holmes, J. Org. Chem. 1994, 59, 7955; B. J. Egner, G. J. Langley, M. Badley, J. Org. Chem. 1995, 60, 2652).

In most coding procedures further compounds which are specific for each synthesis module are coupled to the polymer in addition to the synthesis modules which have been/will be coupled prior or after coupling the latter. Thus, these marker substances, which are designated as “tags”, uniquely archive the timing sequence of the synthesis. Such a coding systems has, however, the decisive disadvantage that it has to be compatible to the used chemism, that is, the chemist is not free in his/her approaches to the syntheses. Moreover, the analysis, that is, the decoding of the chemical tags, requires high logistic expenditures (S. Brenner, R. A. Lerner, Proc. Natl. Acad. Sci. 1992, 60, 5381./Z. J. Ni, D. MacLean, C. P. Holmesm, M. M. Murphy, B. Ruhland, J. W. Jacobs, E. M. Gordon, M. A. Gallop, J. Med. Chem. 1996, 39, 1601./a) M. H. J. Ohlmeyer, R. N. Swanson, L. W. Dillard, J. C. Reader, G. Asouline, R. Kobayashi, M. Wigler, W. C. Still, Proc. Natl. Acad. Sci. 1993, 90, 10922. b) H. P. Nestler, P. A. Bartlett, W. C. Still, J. Org. Chem. 1994, 59, 4723).

Hence, the usual combinatorial “Split and Mix” procedures are the quickest way in solving the problem of the combinatorial variety. However, the procedures of the “chemical tagging” are strongly restricted by the considerable logistic expenditures after completion of the synthesis.

In order to solve this problem, a physical way is chosen with the so-called “IRORI radio tag” method, and this on a macroscopic basis: Hereby a so-called “radio-frequency tag”, shortly, an RF-tag, which is embedded in glass, is additionally inserted into a micro-reactor which is filled with polystyrene spheres. The RF-tag substantially consists of an antenna and a transmitter and a receiver, respectively. Each chemical synthesis step und the corresponding unit made up of the reactor and the RF-tag are uniquely characterized by encoded numbers. The respective numbers can be both, read and written. In this way the procedure is completely described. The RF-tag method is disadvantageous due to the comparatively high cost and also to the lacking possibility to miniaturize the method without high expenditures.

Furthermore, there are miniaturized methods known. With the so-called AFFYMAX-technique with position specific light induced coupling reactions (S. P. A. Fodor et al. (1991) Science 767-773) a specific compound can be uniquely determined by its respective x/y-position. The method, however, is restricted in its applicability for diverse reasons. It is, for example, restricted to photo-activatable coupling reactions and requires extensive intermediate steps and washing steps at each reaction since all reaction positions are brought into contact with each of the entire reagents used. Moreover, it has to be considered as disadvantageous that the reactions have to be carried out in sequence when different reagents are used per reaction step.

In R. Frank, Tetrahedron Vol. 48, 42, 9217-9232, a method is described which permits to make up libraries of solid-phase coupled peptides in parallel performed synthesis on cellulose filters in a spot procedure. The libraries are subsequently submitted to a functional examination, for example, to a binding to an antibody. Comparable methods have been described by Fodor et al. (Science 1991, 251, 767-773) and Geysen et al. (Proc. Natl. Acad. Sci. 1984, 81, 3998-4002). Further methods will be discussed in an overview (Fields et al. Int. J. Peptide Protein Res. 1990, 35, 161-214). As to the entire methods involved and apart from some variations, the coupling chemistry is on principle comparable and falls back on traditional methods with respect to solvents, protective group chemistry and activation. Comparable methods have been developed for the oligonucleotide synthesis. The outputs naturally are low and are not accessible any more to a macroscopic characterisation.

In summing up it has to be said about the prior art that the parallel synthesis identifies the respective resulting product by way of the coordinates of the respective reaction vessels (or of the spots in the case of miniaturized areal methods). In contrast thereto, a subsequent determination of the product identity is required with the combinatorial synthesis, unless there are provided uniquely determinable physical encodings, as is the case with the IRORI-radio-tag method. These known methods exhibit the disadvantages described herein above.

### SUMMARY OF THE INVENTION

It is an object of the present invention to provide a method for carrying out chemical or biological reactions or syntheses and to functional particles suited for these, which ensures a complete compatibility between micro scale and macro scale, whereby a great variety of coupling reactions can be carried out and whereby a unique coordination of the reaction or synthesis products to the individual functional particles can be ensured at low expenditures.

### DETAILED DESCRIPTION OF THE INVENTION

The object is realized by the features of the first and the fourth claim. In the following, the invention will be explained in more detail by virtue of schematical embodiments. There is shown in:

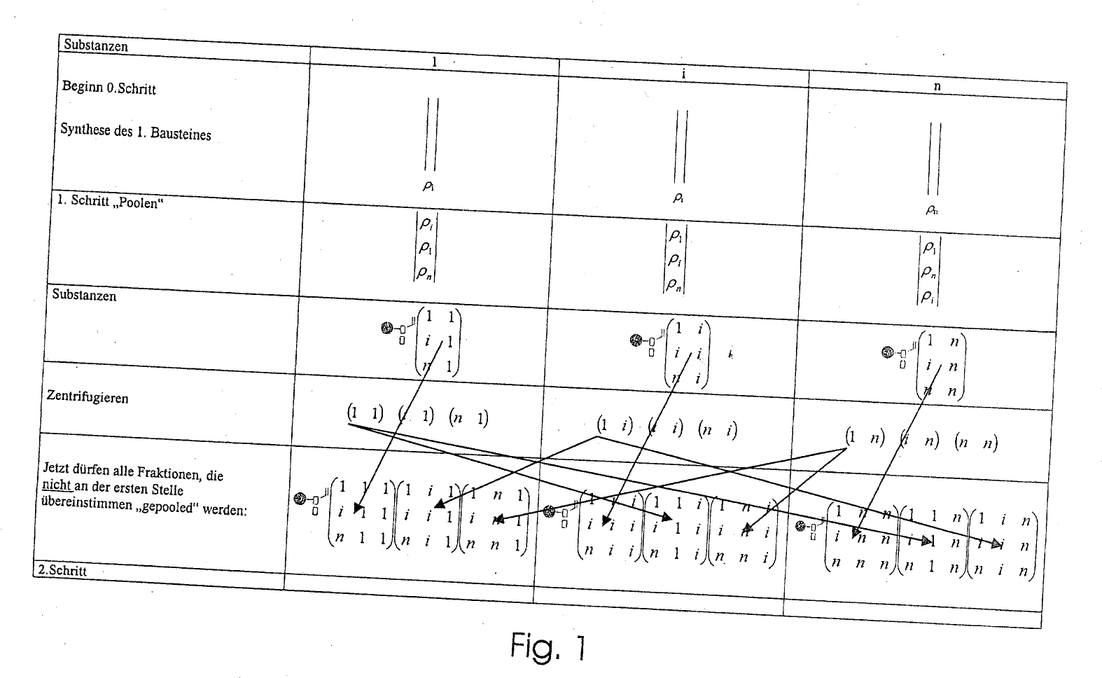

FIG. 1 a part of the sequence according to the method for n synthesis reaction vessels and n functional particles, and

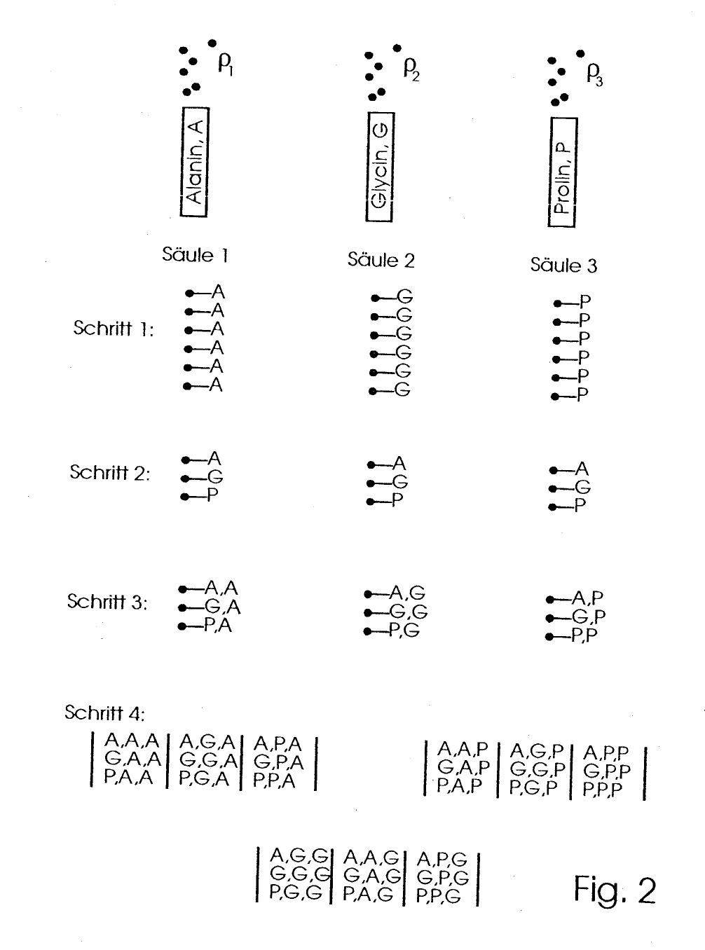

FIG. 2 an exemplary special procedure of method for formation of a peptide library.

In a first embodiment according to FIG. 1 there are provided in an exemplary fashion n synthesis reaction vessels (for example, columns) and n encoded charges of particles. The n particle charges differ from one another in that they exhibit densities p_{1 . . . n} which differ from one another, whereby the density represents the code. In this way any desired substance classes≦n permits synthesizing as follows:

In the example, each of the n reaction vessels shall be supplied with respective different chemical reaction solutions from the pool of modules to be combined (see FIG. 1). Then an equal number, if possible, of functional beads are provided in each column, whereby to each column functional beads of like density are allotted, whereby the densities of the functional beads which are added to the individual columns differ from each other.

The first coupling step is carried out in allocating a given amount of functional particles of type 1 to the reaction vessel 1, of the type i to the reaction vessel i, up to the type n to the reaction vessel n. The synthesis coupling (different solvents, protocols etc.) is carried out. Then the functional beads are taken from the reaction vessels, the respective charges distributed according to the number of further reactions to be carried out, in the example to n equal parts, and combined (mixed) to yield new charges in such a manner that a mixture of particles of n different densities results per charge, that is, for example, in the i-th column there are proportional charges of all n coded particles and, hence, n different substances. Accordingly, in a next step of the synthesis there are generated in the column i all combinations from (1 . . . i . . . n)·i. This library can now be separated into its respective individual components via a sedimentation procedure or by centrifugation. Hereby, it is only important that the coordination to the respective step and to the respective column, respectively, is maintained: hence, ((1,i); . . . (i,i) . . . (n,i)) are filled each into vessels and the numbering and the coordination registered separately.

In the subsequent step all fractions which do not match in their first position will now be combined with one another, that is, a part of the initial fraction will be combined with i ((1,i); . . . (i,i); . . . (n,i))·i=((1,i,i); . . . (i,i,i); . . . (n,i,i)); a part of the fraction, for example, of the first column will be combined in a same manner with i ((1,1); (i,1); (n,1))·i=((1,1,i); (i,1,i); (n,1,i) etc.

Thus, this method permits to generate all possible substance combinations, whereby only some caution has to be exercised between the single steps in order to avoid combinations of particles having like density and a similar history.

The above described method of proceeding of separating by particle densities after completion of a synthesis step and a further combination of charges has to be carried out as long until the desired reaction chain lengths have been obtained, respectively, as long until only one respective particle per preselected different density is obtained per combined charge. Depending on the desired synthesis and on the number of submitted reaction solutions, a separation of the combined charges is not necessarily required after each synthesis step, as will become apparent from the following embodiment.

In this second embodiment a simple case of a peptide library, and for the sake of simplicity, only three fractions will be described, (whereby the above method will by no means be restricted to peptide or other oligomers):

As shown in FIG. 2, three columns are provided which are each charged with particles of different density ρ₁, ρ₂, ρ₃. In the example, alanine A shall be bonded to the respective polymer beads in the first column, glycine G in the second column, and proline P in the third column. After taking out the polymer beads (step 1), a division of the respective charges into three is carried out and mixing them together to yield three further charges which contain respective polymer beads of all three initial charges of different densities (step 2). In the example, these charges are added, in turn, to the respective three columns 1 to 3 so that the syntheses A, A; G, A and P, A are carried out in the first column, the syntheses A, G; G, G and P, G are carried out in the second column, and the syntheses A, P; G, P and P, P are carried out in the third column. The result of the proceeding is schematically shown in FIG. 2, step 3. In the example, the three charges obtained in this manner shall be divided statistically, that is, without a splitting up according to the different densities of the polymeric beads, into three parts each and the columns 1 to 3 will each be charged, again, with one respective part. The variety of syntheses obtained in this manner including all possible combinations of substances is represented in step 4 of FIG. 2. Since this variety complies with the chain lengths desired in the example, a splitting up by density of the polymer beads only occurs after the last step of the synthesis. Since the individual synthesis steps are protocolled it is possible in a simple fashion to exactly associate the particles and the chains synthesized thereto.

The separation of the synthesized substances via density encoding can be carried out with high precision. Ultra-centrifuges are capable of even separating, for example, two different DNA-strands which only differ by the natural positions of the ¹⁴N by the isotope ¹⁵N. The separation of complementary λ-phages DNA-strands is also possible: 1.743 and 1.730 g/cm³. In the case of greater particles, however, already simple sedimentation procedures will be sufficient. Depending on the separation procedure used and the number of synthesis steps to be carried out, it has only to be cared for within the scope of the invention that the differences in density of the single polymer beads are still great enough when being charged with the synthesis chains to permit an exact separation by density differences. The charging, for example, of a 100 μm polystyrene bead corresponds in about to a 100 pMol synthesis substance. When taking, for example, two polystyrene/composite beads of a diameter of 100 μm and a density of 1 and 1.1 g/cm³, respectively, which corresponds to a weight of about 5.2 and 5.72·10⁻⁷ g, respectively, and a charge of 100 pMol with a mean molecular weight of about 110, which corresponds to a weight of about 10⁻⁸ g, then a density change of about 2% will result. Since the densities with all sorts of beads will change at an average and the main component will remain unchanged the method is broadly applicable.

The main advantage of the present invention in contrast to the described solutions of the prior art consists in that the number of the required synthesis steps is reduced to the number of the density fractions used and in that, at the completion of the synthesis steps, an exact association of the synthesis chains to the single functional beads is given.

SiO₂ particles, for example, obtained by a suspension polymerization are suited as functional beads which are used in the proposed method. Thereby, in the example, 15 g SiO₂ particles having a diameter in a range of from 5-20 μm will react for 90 min. with Methacrylolyl-oxypropyl-trimethoxysilan (0.5 ml dissolved in 30 ml toluol) under moisture exclusion at 40° C. Then the particles will be tried in a rotation evaporator under vacuum at ambient temperature. In a 11-reactor which is provided with a stirrer and a reflux condenser, 650 ml of a solution of 2-g polyvinylpyrrolidone K90, 650 mg CaSO₄, and 100-mg calciumphosphate are initially put in. Thereto, at 78° C., 15 g pretreated silicon dioxide are added suspended in a mixture of 30 ml styrene, 0.6 ml divinyl benzene and 400 mg Dibenzoylperoxyd and suspended at a rotation speed of 500 rpm. After completion of the polymerization after 6 h the composite particles are sucked off, washed and classified by screening. In this manner one gets 43 g of particles of a diameter in the range of from 100 μm to 800 μm and of a density in the range of from 1.00-1.5 g/cm³, from which, after screening and separation by density, the charges of different densities required for the method can be separated. When required and as common use in the prior art, these particles can be provided with anchor groups for a temporary immobilization of the first chemical or biological component.

For bio-analytical applications also particles can be used which are obtained as follows: a melt of polystyrene in toluol and a suspension of silicon dioxide (diameter of particles 5-10 μm) is mixed in a graded mixing chamber and successively dripped under use of methyl alcohol into a cooled distilling receiver. In this way particles are obtained which have a diameter of 150 μm and a density distribution in a range of from 1 g/cm³ to 2.0 g/cm³.

These particles are soluble in a great number of organic solvents, but they are not soluble in water. For example, proteins can be immobilized thereupon and can be used for assays in aqueous media.

Furthermore, it lies within the scope of the invention to use other suited functional particles, even such of different composition or character, provided that they are inert towards the employed reaction solutions and provided that the measures for the different densities of the single charges of particles are satisfied.

1. Method for carrying out chemical or biological reactions or syntheses, whereby a great number of functional particles are used which enables a coupling of chemical components or solid phase syntheses, characterized in that when n reaction solutions are used then x charges of functional particles are provided, whereby x≦n, the density of which being determined differing from one other in such a way that they can be separated by their densities even when charged with the complete reaction or synthesis product, wherein
a) initially each charge of a homogeneous density is added to a first reaction solution, after bonding to a first reaction partner
b) each of said x charges is separated into maximally n partial sets and a mixture of the partial sets with one another to yield respective novel charges containing functional particles of all densities is produced, and
c) said novel charges each being added to a further reaction solution, whereby after completion of the second reaction and synthesis, respectively,
d1)either a statistical distribution of the respective partial charges again into respective maximally n novel subsets is carried out, or
d2)at least once a separation by density of the partial charges obtained is carried out and the respective subsets of different origin again are mixed with one another to yield novel partial charges, and
e) the steps c), d1) or d2) are repeated until the desired reaction products or synthesis products are obtained or, per subset, no less than one functional particle each of the n different densities used is obtained, whereby
the reaction or synthesis path of the functional particles in each of the above steps of the method is protocolled.

2. Method as claimed in claim 1, characterized in that, when carrying out a plurality of reactions, the obtained partial charges of functional particles of different densities are repeatedly separated by their density and the subsets obtained in this way are combined to novel partial charges.

3. Method as claimed in claim 1, characterized in that when carrying out x reactions, the obtained partial charges of functional particles of different densities are separated by their density after each reaction and the subsets obtained in this way are combined to novel partial charges.

4. Functional particles for carrying out chemical or biological reactions or syntheses according to one of the preceding claims, characterized in that in dependence on the number n of presentable reaction solutions x respective charges of a respective greater number of functional particles are provided, whereby x≦n, whereby the densities of the individual charges being determined such great differing from one other in such a way that, even when charged with the complete reaction or synthesis product, the functional particles can be separated by their density by way of suitable separation methods.
